(19) United States
(12) Patent Application Publication
(10) Pub. No.: US 2022/0223256 A1
(43) Pub. Date: Jul. 14, 2022

(54) Diet System With The Use Of Cards

(71) Applicant: Michail Makrynakis, Foothill Ranch, CA (US)

(72) Inventor: Michail Makrynakis, Foothill Ranch, CA (US)

(21) Appl. No.: 17/646,721

(22) Filed: Jan. 1, 2022

Related U.S. Application Data
(60) Provisional application No. 63/136,674, filed on Jan. 13, 2021.

(51) Int. Cl. G16H 20/60

(52) U.S. Cl. CPC G16H 20/60

(57) ABSTRACT

A diet system that uses cards to manage meal plans is disclosed. The system comprises of meal cards with meal plans. The cards are split into different categories such as breakfast, snacks, dessert, main meals and detox, among others, wherein cards in a category have an identifying characteristic such as color. Each card has a day's meal plan for the category and may further include various meal choices. The system is used in weight and diet management, wherein meals are spontaneous and offer the user a more flexible diet option.

### FIELD OF INVENTION

In general, the present invention relates to the use of meal cards for weight and diet management. More specifically, the invention relates to a diet system that uses cards to manage meal plans, wherein cards are split into different categories such as breakfast, snacks, dessert, main meals and detox, among others, with cards in a given category having an identifying characteristic such as color.

### CROSS-REFERENCE TO RELATED APPLICATIONS

This application claims the priority benefit of the provisional application No. 63/136,674 filed on 13 Jan. 2021.

In some embodiments thereof, the current invention benefits from several inventions published before. They include:

U.S. Pat. No. 4,606,555: A diet control device and method for use by a dieter in administering a diet plan of particular food items which includes a booklet having a first cover, a second cover foldable relative to the first cover, and a plurality of cards attached to one of the covers, with each card being marked to represent a different day in the diet plan. Each of the cards includes a plurality of slits to form a plurality of individually removable and disposable tabs or splints.

U.S. Pat. No. 4,976,622: A system to aid a person in learning a prescribed diet, including food groups, allowed food portions, prescribed meal and medication schedule in which a two panel folder is provided, each panel having a set of pockets labeled for each meal and snack of a day of the diet.

U.S. Pat. No. 7,213,743: Methods 300 and systems 100 for evaluating proposed food purchases which vary from a pre-planned diet are provided. A nutrition-related scheme, the pre-planned diet, is developed on the basis of achieving a primary goal such as increasing the intake of a particular nutrient or losing a certain amount of weight. The nutrition-related scheme, which may be stored in a memory 103 of a computer 120, includes a list of approved food items.

AU2011315835: Nutritional compositions and formulations that optimize nutritional contents are provided. Dietary compositions and methods for tailoring such compositions to optimize levels of nutrients that have beneficial effects within specific ranges are provided. Dietary plans, and formulations comprising dietary products that comprise optimized levels of nutrients derived from phytochemicals, antioxidants, vitamins, minerals, lipids, proteins, carbohydrates, probiotics, prebiotics, microorganisms and fiber. Diet plans and modular nutritional packages comprising food and drink items tailored according to consumer patterns typed by

AU2011202842: This business method invention provides financial motivation to individuals for attaining and maintaining weight loss goals. It requires individuals to compete against other individuals for a prize made up from the sum of contestants' monetary stakes.

CN207148958: The utility model provides a dining room dining room management system of health control belongs to the intelligent control field, and the system includes PC, the meal card machine of supplementing with money, menu reading and writing platform, built-in radio-frequency card tableware, the card of having a dinner, intelligent settlement platform, server and subscriber equipment.

CN109285596: The invention discloses a diet recommendation system and method for a smart family. The diet recommendation system comprises a diet recommendation device and a cloud server; the diet recommendation device performs data information transmission with the cloud server through a network;

U.S. Pat. No. 5,451,079: A universal diet card for providing dietary information in hospitals or similar facilities and a related method for the use of the universal diet card are disclosed in which a single diet card is usable for any of a plurality of special diets and is capable of containing extensive information relative to the desired diet for an individual.

U.S. Pat. No. 5,338,202: This invention relates to a daily diet management planner and method comprising a foldable carrying case having a plurality of pockets and pocket inserts on and in the inside walls of the carrying case: having meal insert cards each identifying a specific meal type such as BREAKFAST, MORNING SNACK, LUNCH, AFTERNOON SNACK, DINNER, and BEDTIME SNACK; and having a plurality of food cards each listing one specific food within a food exchange and displaying a picture of either the approximate or visual serving size of the food.

US20050048454: A diet planner and method providing a card showing a plurality of food item lists and the portion permitted, wherein the food item lists are divided into categories and each category name presents a characteristic color. The combination of three or more categories creates a food group called “asterisk” which maintains stable the sugar levels (glucose) in the blood.

### BACKGROUND OF THE INVENTION

There are hundreds of fad diets, weight-loss programs and plans that promise quick and easy weight loss. However, the foundation of successful weight loss remains a healthy, calorie-controlled diet combined with increased physical activity. For successful, long-term weight loss, you must make permanent changes in your lifestyle and health habits.

The principle of weight gain is simple: energy intake exceeds energy expenditure. However, overweight and obesity are clearly the result of a complex set of interactions among genetic, behavioral, and environmental factors. While hundreds, if not thousands, of weight-loss strategies, diets, potions, and devices have been offered to the overweight public, the multi-factorial etiology of overweight challenges practitioners, researchers, and the overweight themselves to identify permanent, effective strategies for weight loss and maintenance. The percentage of individuals who lose weight and successfully maintain the loss has been estimated to be as small at between 1 to 3 percent.

The use of behavior and lifestyle modification in weight management is based on a body of evidence that people become or remain overweight as the result of modifiable habits or behaviors, and that by changing those behaviors, weight can be lost and the loss can be maintained. The primary goals of behavioral strategies for weight control are to increase physical activity and to reduce caloric intake by altering eating habits.

This disclosure presents a diet system that uses cards as a practical tool that can help reach weight-loss and weight management targets.

### SUMMARY OF THE INVENTION

The following summary is an explanation of some of the general inventive steps for the system, method, architecture and tools in the description. This summary is not an extensive overview of the invention and does not intend to limit the scope beyond what is described and claimed as a summary.

According to some embodiments, the invention relates to a diet system that uses cards to manage meal plans. The system comprises of meal cards with meal plans. The cards are split into different categories such as breakfast, snacks, dessert, main meals and detox, among others, wherein cards in a category have an identifying characteristic such as color. Each card has a day's meal plan for the category and may further include various meal choices. The system is used in weight and diet management, wherein meals are spontaneous and offer the user a more flexible diet option. While there are many solutions that use coded cards, the prior art calls for a method and apparatus for pre-selecting the cards, causing monotony and resulting in a greater likelihood of a user going off of his/her diet, a problem that the current invention seeks to solve.

According to one embodiment, the user would not preselect a meal plan but instead draw each card only as needed, adding an element of spontaneity. This distinction may allow us to overcome the prior art whereby an entire day's meal plan is pre-selected causing monotony and resulting in a greater likelihood of a user going off of his/her diet.

According to one embodiment, the user may select several cards to cover all the days meal plans including breakfast, lunch, dinner, snacks among others.

According to one embodiment, a user may select a single card which has several plans to cover some or all the days meal plans including breakfast, lunch, dinner, snacks among others.

According to one embodiment, the meal card may be provided in a digital format encoded in a portable computing device.

According to one embodiment, meal cards may be completely operable without physical cards, or a combination of physical cards and digital formats in computing devices.

### BRIEF DESCRIPTION OF THE FIGURES

The novel features believed to be characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 of the diagrams illustrates a meal card, wherein the illustrated meal card has a meal category and a meal plan.

FIG. 2 of the diagrams is an illustration of a card system designed such that for a meal category for example breakfast, there is one or more cards to choose from, each bringing uniqueness to the plan.

FIG. 3 of the diagrams demonstrates a front and back of a card, where there is a meal plan at the front and at the back to offer the user more choices over their planned diet.

FIG. 4 of the diagrams is an array of meal cards, which together form a complete plan as may be determined from the available categories.

FIG. 5 of the diagrams is an illustration of the current invention in a digital format as provided in a portable computing device.

### DETAILED DESCRIPTION OF THE INVENTION

Hereinafter, the preferred embodiment of the present invention will be described in detail with reference to the accompanying drawings. Noteworthy, the embodiments disclosed are only examples and this disclosure is not limited to these specific examples. Embodiments described in the following description may be incorporated into other systems and methods not disclosed in the following description. Structures, elements and process steps shown in the drawings are exemplary embodiments and are not to be used to limit broader teachings.

It is understood through the text of this disclosure that where elements or process steps are described as separate units, those skilled in the art will recognize that various elements or portions thereof may be integrated together. Where elements are described in the following description as integrated together into a combined element, those skilled in the art will similarly recognize that individual elements of the combination may be utilized as separate elements.

This specification includes reference to “an embodiment” or “one embodiment”. This language is intended to refer to the particular elements, structures and process steps of the embodiment being discussed in that portion of the specification. Where references are made to “an embodiment” or “one embodiment” in other portions of the specification, those similarly refer those particular elements, structures and process steps of the embodiment being discussed in that portion of the specification. Embodiments discussed in different portions of the specification may or may not refer to the same embodiment.

The use of specific terminology in the specification is used for best describing the invention and shall not be construed as limiting. The terms “include”, “including”, “comprise” and “comprising” shall be understood to be open terminology and not limiting the listed items.

The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms in order to describe the invention in the best way.

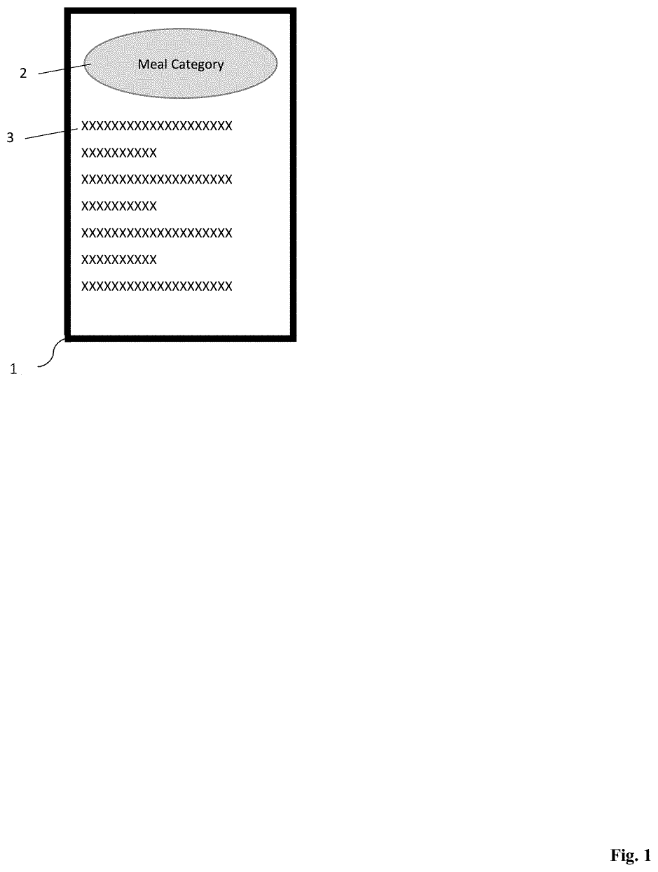

In a first embodiment according to FIG. 1 of the diagrams, it is illustrated a meal card 1, wherein a meal card has a meal category 2 and a meal plan 3. In the context of the current invention, a meal plan can be anything that is food-related such as vegetables or cereals, and how such are to be used in a prescribed portion.

The meal category 2 may comprise breakfast, snacks, dessert, main meals, lunch, dinner and detox, among others and in any such combinations as may be necessary to meet the objectives of the user. For example, in one exemplary illustration, the categories may include breakfast, lunch, dinner and snacks. In the exemplary illustration above, the categories may comprise cards with a variety of meal plans 3, such as:

### Breakfast

Card 1: Bacon, eggs, toast and milk
Card 2: Cereals, milk and banana
Card 3: Fresh orange juice, pancake and 2 sausages
Card 4: Donut, tea and an orange
Card 5: Muffin, cookie, milk and peanut butter
Main meals (lunch and dinner)
Card 1: Green bean casserole, mushroom soup and fried onion straws
Card 2: Hamburger and potato fries
Card 3: Mashed potatoes and lamb chops
Card 4: Beef steak and boiled plantains
Card 5: Spaghetti and meatballs
Card 6: White rice and barbecue ribs

### Snacks

Roast peanuts, potato crisps, pop corn, chocolate, cheese sticks, cookies, corn chips, biscuit, roast pumpkin seeds.

The examples listed there are for purposes of explaining the invention and are not an endorsement for healthy dieting. For example, there may be five cards to select breakfast from, six cards for lunch (lunch in this particular case is considered a main meal), six cards for dinner (again considered a main meal) and nine cards for snacks. The user may, for their dieting purpose select a breakfast card, a lunch card, a dinner card and a snack card. The cards will guide the user on their meal plans for the day, for the planned meals. For example, an exemplary plan may comprise a user having breakfast, lunch, dinner and snacks at proposed intervals throughput the day. The plan may further have portions for each category defined, for example, the lunch may serve a smaller portion than dinner, or any such order.

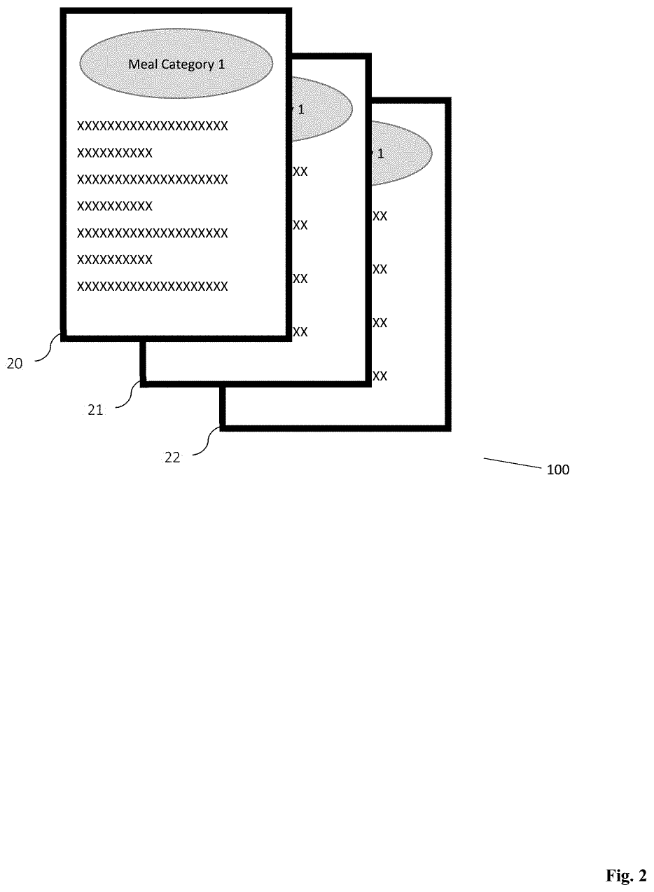

In a second embodiment according to FIG. 2 of the diagrams is an illustration of a card system 100 designed such that for a meal category 1, for example breakfast, whereby there is at least a card to choose from and where there are several cards shown as 20, 21 and 22 to choose from, each bringing uniqueness to the plan. For example, the first card 20 may prescribe a breakfast of bacon, eggs, toast and milk, the second card 21 prescribing fresh orange juice, pancake and 2 sausages, while the third card 22 prescribes a muffin, cookie, milk and peanut butter. Important to note is that the cards in a category have the same identifying characteristic preferably a color, but it could also be the shape or any number of characteristics that can help make a card as unique. According to this embodiment, there may be as many cards for a defined category as is necessary to offer maximum flexibility to the user. Once selected, the user shall have their meals according to the plan of the selected card, and in the portions defined therein.

It is anticipated that a meal category may be split into several sub-categories, each sub-category having select-able cards.

It is also anticipated that for each category, the meal plan may be a combination of several select-able sub-categories.

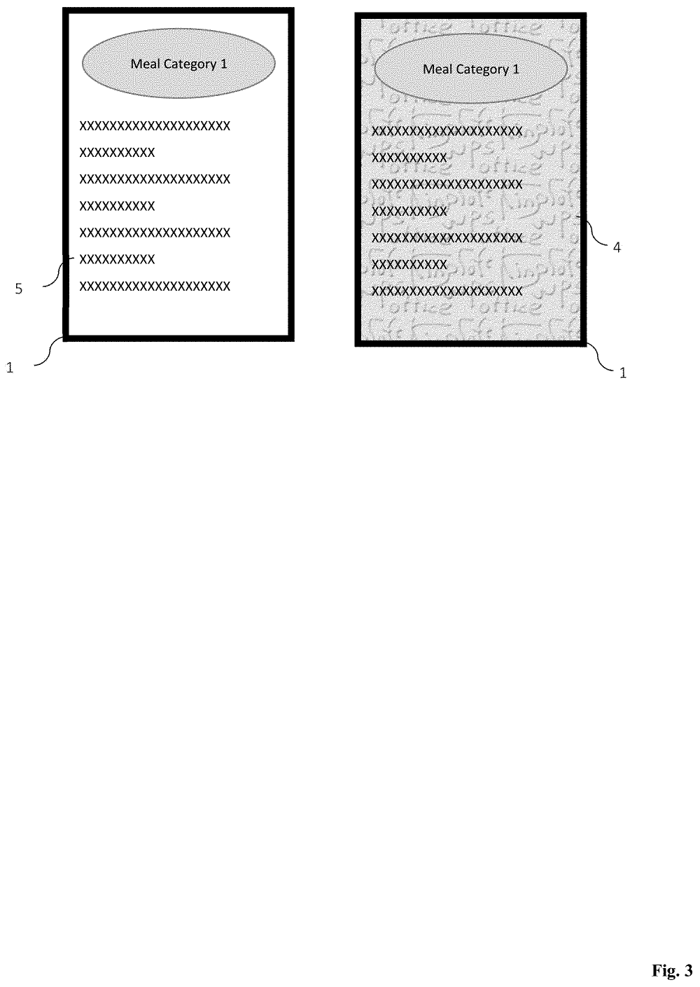

Further in FIG. 3 of the diagrams demonstrates a front 5 and back 4 of a card 1, where there is at least a meal plan at the front and at the back to offer the user more choices over their planned diet. A typical cardholder can choose whatever card groups they want to based on the characteristics of a category they want to go with on that day. But the important thing is to continue with the same group until the end of the day so that the diet is successful. In case they have chosen two cards that displays the same meals, they just need to pick another one for them to be different.

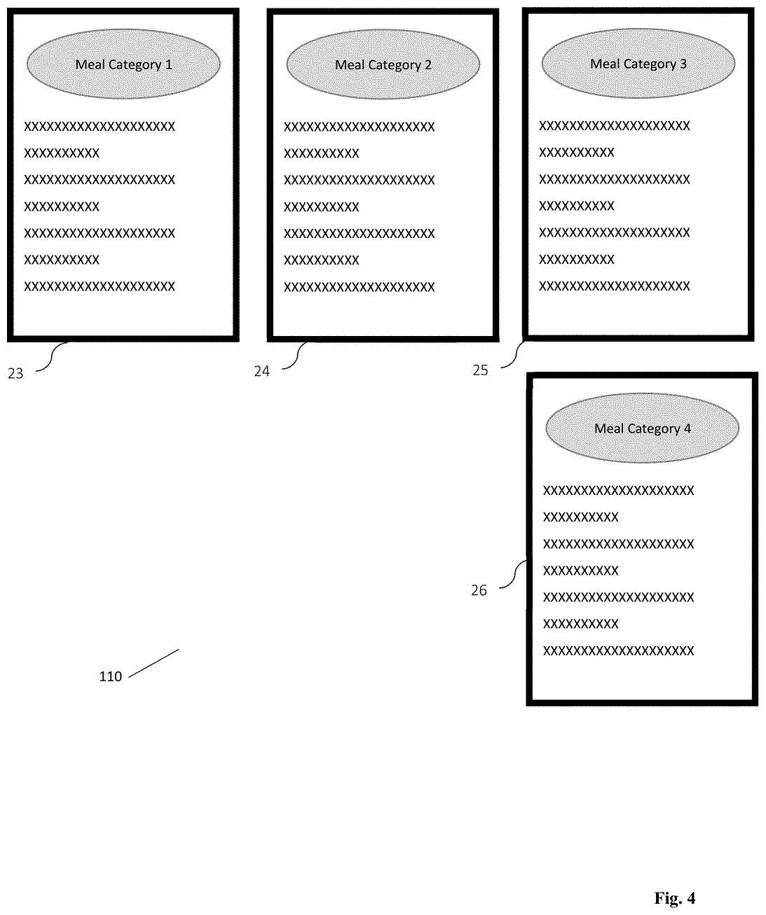

In the embodiment according to FIG. 4 of the diagrams is an array 110 of meal cards 23, 24, 25 and 26 which together form a complete plan as may be determined from the available categories. As an example, with the system, everyone can create a diet plan with five meals per day or more by merely shuffling the cards and choosing between cards. If their choices have the same meals, they can keep one option and choose another meal to be different. Inherently, most dieters do not enjoy having someone controlling their daily, weekly, monthly food choices. So they take their diet into their own hands. But the problem is they tend to go hard on themselves, removing entire food categories and going into starvation. This tactic usually works in the short term but rarely lasts. Often, people gain the weight they lost back soon after “going off” their diet. But with the current invention, all meals are displayed in cards so that everyone can make their diet plan, without the stress of having someone controlling their food, but also ensuring that they follow a balanced diet without risking their health and achieve weight management at the same time.

In the above example, a user may select a combination of:
On the first day:
Breakfast, 23: Fresh orange juice, pancake and 2 sausages
Lunch, 24: Spaghetti and meatballs
Dinner, 25: Green bean casserole, mushroom soup and fried onion straws
Snacks, 26: Roast peanuts
On the second day:
Breakfast, 23: Muffin, cookie, milk and peanut butter
Lunch, 24: Hamburger and potato fries
Dinner, 25: White rice and barbecue ribs
Snacks, 26: Cheese sticks
On the third day:
Breakfast, 23: Bacon, eggs, toast and milk
Lunch, 24: Mashed potatoes and lamb chops
Dinner, 25: Beef steak and boiled plantains
Snacks, 26: Roast pumpkin seeds

In the example combination above, it is demonstrated that cards can be combined in a variety of exciting ways by the user to fulfil their day's meal plans. However, it is to be noted that the above example is exemplary as a complete meal play may comprise any number of categories and combinations, including those not explicit described in this disclosure. Nonetheless, even in novel combinations in whatever combinations they are required, for example six, seven or any number of meals a day, may also be performed as described by having each of the categories for the six, seven or any number of meals a day having a category with select-able cards that a user of the system may use.

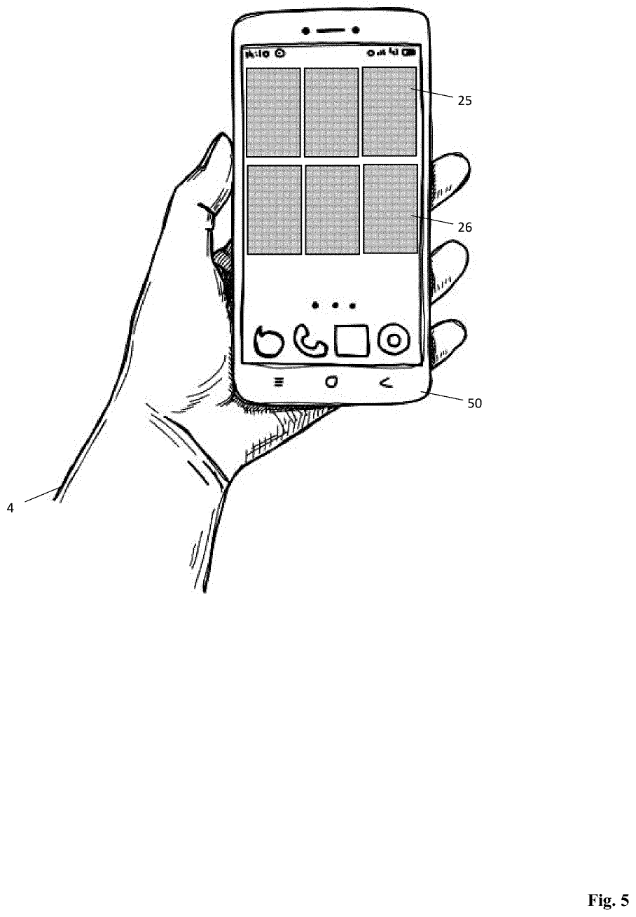

In a non-limiting embodiment according to FIG. 5 of the diagrams is an illustration of the current invention in a digital format as provided in a portable computing device. A portable computing device 50 is shown in the figure as held by the hand 4 of a person using the current invention. The preferable type of device used in the illustration comprises at least one processor, a memory, a network communication port, a display and input devices, which may include keyboards, mouses, haptic touch devices or any means of providing instructions into a portable computing device. The memory of the device is encoded with instructions, executable by the processor as to render on the graphical user interface meal cards such as 25 and 26 shown for the selection of a user. It is anticipated that the device may be operably coupled top a remote computer that may be responsible for generating said meal cards and a network to enable their transmission to the portable computing device.

It is also anticipated that the device 50 may be a standalone device fully capable of generating the meal cards, managing their selection by the user and availing them on the display.

It is anticipated that the digital format of the invention may disclose a distributed system architecture, an event-driven architecture, a service oriented architecture, a complex system architecture with a plurality of devices configured to pass messages between each other, and adapted towards a common function or a monolithic system architecture.

As with physical card system, the digital card system enables everyone to make their diet plan using digital cards on the computing devices, freely and while ensuring that they follow a balanced diet without risking their health and achieve weight management at the same time.

As with a physical card system used in the exemplary embodiments above, in the the digital system of this embodiment, there may be five cards to select breakfast from, six cards for lunch (lunch in this particular case is considered a main meal), six cards for dinner (again considered a main meal) and nine cards for snacks. The user may, for their dieting purpose select a breakfast card, a lunch card, a dinner card and a snack card. The digital cards will guide the user on their meal plans for the day, for the planned meals. As with the examples above, and as an example, an exemplary plan may comprise a user having breakfast, lunch, dinner and snacks at proposed intervals throughput the day as operated in the computing device. The plan may further have portions for each category defined, for example, the lunch may serve a smaller portion than dinner, or any such order.

As with physical cards, it is important to note is that the digital cards in a category have the same identifying characteristic preferably a color, but it could also be the shape or any number of characteristics that can help make a card as unique. According to this embodiment, there may be as many cards for a defined category as is necessary to offer maximum flexibility to the user. Once selected, the user shall have their meals according to the plan of the selected card, and in the portions defined therein.

It is anticipated that a meal category may be split into several sub-categories, each sub-category having select-able cards.

It is also anticipated that for each category, the meal plan may be a combination of several select-able sub-categories.

In the example combination above, it is demonstrated that cards can be combined in a variety of exciting ways by the user to fulfil their day's meal plans.

Although a preferred embodiment of the present invention has been described for illustrative purposes, those skilled in the art will appreciate that various modifications, additions and substitutions are possible, without departing from the scope and spirit of the invention as disclosed in the accompanying claims. Such alterations are herewith anticipated.

### INDUSTRIAL APPLICATION

The current invention technology useful in improving meal cards for weight and diet management.

The invention is also applicable to diet planning systems and implements.

What is claimed is:

1. A diet system that uses cards, the system comprising of:
cards grouped into categories of preferred meals, wherein:
cards in the same category have the same identifying physical characteristic, and;
each card has an identifying category on it, and a meal plan for the identifying category.

2. The diet system as in claim 1, wherein the identifying characteristic is color.

3. The diet system as in claim 1, wherein the identifying characteristic is shape.

4. The diet system as in claim 1, wherein card categories are further divided into sub-categories.

5. The diet system as in claim 4, wherein several cards in a sub-category are combined for a complete meal plan in the category.

6. The diet system as in claim 1, wherein the category may be at least one of breakfast, snacks, dessert, main meals and detox.

7. A computer program product configurable on the memory of a computing device, and executable by at least a processor, the execution of which causes the implementation of a digital cards diet system comprising of:
cards grouped into categories of preferred meals, wherein:
cards in the same category have the same identifying physical characteristic;
cards are displayed on the display of the computing device, and;
each card has an identifying category on it, and a meal plan for the identifying category.

8. The computer program product as in claim 7, wherein the identifying characteristic is color.

9. The computer program product as in claim 7, wherein the identifying characteristic is shape.

10. The computer program product as in claim 7, wherein card categories are further divided into sub-categories.

11. The computer program product as in claim 10, wherein several cards in a sub-category are combined for a complete meal plan in the category.

12. The computer program product as in claim 1, wherein the category may be at least one of breakfast, snacks, dessert, main meals and detox.

13. A method of providing a diet system that uses cards, the method comprising of:
providing cards grouped into categories of preferred meals, wherein:
cards in the same category have the same identifying physical characteristic, and;
each card has an identifying category on it, and a meal plan for the identifying category.

14. The method as in claim 13, wherein the identifying characteristic is color.

15. The method as in claim 13, wherein the identifying characteristic is shape.

16. The method as in claim 13, wherein card categories are further divided into sub-categories.

17. The method as in claim 17, wherein several cards in a sub-category are combined for a complete meal plan in the category.

18. The method as in claim 13, wherein the category may be at least one of breakfast, snacks, dessert, main meals and detox.

19. The method as in claim 13, further comprising providing a computer program product configurable on the memory of a computing device, and executable by at least a processor, the execution of which causes the implementation of a digital diet system that uses cards.

20. The method as in claim 19, wherein cards are displayed on the display of the computing device.
